(19) United States
(12) Patent Application Publication
(10) Pub. No.: US 2014/0135245 A1
(43) Pub. Date: May 15, 2014

(54) CLEANING COMPOSITION

(71) Applicants: Stephanie Annaheim, Burnhaupt-le-Haut (FR); Bruno Bret, Wintzenheim (FR); Herve Walgenwitz, Rixheim (FR); Remy Ruppel, Durrenentzen (FR); Anne-Gaelle Clermont, Colmar (FR); Pierre Probst, Ammerschwihr (FR); Sebastien Jeannot, Holtzwihr (FR)

(72) Inventors: Stephanie Annaheim, Burnhaupt-le-Haut (FR); Bruno Bret, Wintzenheim (FR); Herve Walgenwitz, Rixheim (FR); Remy Ruppel, Durrenentzen (FR); Anne-Gaelle Clermont, Colmar (FR); Pierre Probst, Ammerschwihr (FR); Sebastien Jeannot, Holtzwihr (FR)

(73) Assignee: SCA TISSUE FRANCE, Bois-Colombes (FR)

(21) Appl. No.: 14/128,701

(22) PCT Filed: Jun. 22, 2012
(86) PCT No.: PCT/IB2012/053170

§ 371 Date: Dec. 23, 2013

(30) Foreign Application Priority Data
Jun. 24, 2011 (EP) 11171397.0

(51) Int. Cl. A61K 8/04; A61Q 19/10

(52) U.S. Cl. CPC A61K 8/046; A61Q 19/10
USPC 510/137; 222/190

(57) ABSTRACT

A composition for cleaning, in particular for cleansing of the skin and/or mucous membranes, in particular a personal care cleansing composition, to be applied onto tissue paper, in particular toilet paper, and uses thereof. The composition including at least 70% by weight of water, 0.1 to 1% by weight of at least one gelling agent, and 0.1 to 10% by weight of at least one surfactant including at least one foaming surfactant, relative to the total weight of the composition.

### CROSS-REFERENCE TO PRIOR APPLICATION

This application is a §371 National Stage Application of PCT International Application No. PCT/IB2012/053170 filed on Jun. 22, 2012, which claims priority to European Patent Application No. 11171397.0 filed on Jun. 24, 2011, both of which are incorporated herein in their entirety.

### TECHNICAL FIELD

The present disclosure relates to a composition for cleaning, in particular for cleansing of the skin and/or mucous membranes, in particular a personal care cleansing composition, to be applied onto tissue paper, in particular toilet paper. The present disclosure also relates to the use of said composition.

### BACKGROUND

Improving personal care hygiene, when using the bathroom, is a constant need. Regular and effective cleaning not only improves the level of cleanliness but may also prevent irritation and/or, in some cases, prevent occurrence of infections and/or promote healing of disorders. Thorough cleanliness of the desired surface can be obtained by the conventional water/soap/drying routine. However, when travelling or working away from home, such method is not very convenient.

An alternative is the use of wet wipes or wet folded toilet paper under the faucet. However, these methods are not necessarily the most effective: plain water tears the toilet paper often making it unusable and wet wipes must be kept moist and hence may require specific packaging. Moreover, the waste created with soiled wet wipes, cannot be flushed like toilet paper as they do not disintegrate rapidly in water and can clog the pipes.

It is thus highly desirable to improve personal care hygiene when using bathroom, by ensuring thorough cleanliness of the desired body surface by eliminating the need of the above-mentioned methods.

Foamable cleansing compositions are good alternatives to previously mentioned methods. Such compositions have already been described in DE 10 2009 013 268. A foamable cleansing composition, Aaah® commercialized by Aaah Company, is currently on the market. This composition essentially contains water, a foamable surfactant, an emollient, a soothing agent, a deodorant and natural extracts. Despite their improvements over the previously mentioned methods, these foamable compositions have some drawbacks. The main drawback of these compositions is the insufficient stability of the foam over time once applied onto the tissue paper, causing a too quick absorption of the foam by the paper, and hence a too quick “soak through” and tear of the paper by the foam.

The term “soak through paper properties” refers to the ability of the composition to soak, penetrate, diffuse and pass/go through the sheets/layers of the tissue paper piled on each other. The faster a composition soaks, penetrates, diffuses and passes/goes through the sheets of tissue paper, the faster it can tear said sheets, making them unusable.

Therefore, a need currently exists for a composition for cleansing of the skin and/or mucous membranes, which:

has improved soak through paper properties (does not soak through the paper),

has an effective cleansing action,

does not need to be rinsed, and

leaves a clean, soothing and pleasant feel.

More particularly, a need exists for a composition for cleansing of the skin and/or mucous membranes, which, once applied onto the tissue paper, more particularly toilet paper, is capable of remaining on the surface of the paper in the form of a foam and not soak through the paper during use, thereby allowing efficient cleansing.

In addition, a need exists for a cleansing composition as indicated above that is environmentally-friendly, without extra waste, easy to handle and that does not require costly ingredients.

Furthermore, a need exists for a composition for cleansing of the skin and/or mucous membranes, that is capable of producing high quality foam (high stability over time and good soak through paper properties), with a very good cleansing action, good skin tolerance and skin care action, and without needing to be rinsed.

### SUMMARY

In a first embodiment, the present disclosure addresses these and other needs in the art by providing a composition including:

at least 70% by weight of water, relative to the total weight of the composition;

0.1 to 1% by weight of at least one gelling agent, relative to the total weight of the composition, and

0.1 to 10% by weight, of at least one surfactant including at least one foaming surfactant, relative to the total weight of the composition.

In particular embodiments, the composition satisfies at least one of the following conditions:

once transformed into foam and applied onto the surface of several sheets of tissue paper placed one on top of each other, the time required for said composition to soak through said sheets of paper, with or without applied pressure, is at least two times greater than the time required for a composition without a gelling agent (i.e. comparative examples 1 and 2) to soak through said sheets of paper, and/or

the foam stability of said composition is such that after two minutes, the said composition is transformed into liquid at least two times less fast than a composition without a gelling agent (i.e. comparative examples 1 and 2), and/or

once transformed into foam and applied onto the surface of several sheets of tissue paper, the time required to tear said sheets of paper under a mechanical stress, is at least two times less than the time required to tear said sheets of paper onto which a composition without a gelling agent (i.e. comparative examples 1 and 2) is applied.

In an alternative embodiment, the present disclosure addresses the above-mentioned and other needs in the art by providing a composition including water, at least one gelling agent and at least one surfactant including at least one foaming surfactant, wherein said composition satisfies at least one of the following conditions:

once transformed into foam and applied onto the surface of several sheets of tissue paper placed one on top of each other, the time required for said composition to soak through said sheets of paper, with or without applied pressure, is at least two times greater than the time required for a composition without a gelling agent (i.e. comparative examples 1 and 2) to soak through said sheets of paper, and/or

the foam stability of said composition is such that after two minutes, the said composition is transformed into liquid at least two times less faster than a composition without a gelling agent (i.e. comparative examples 1 and 2), and/or

once transformed into foam and applied onto the surface of several sheets of tissue paper, the time required to tear said sheets of tissue paper under a mechanical stress, is at least two times less than the time required to tear said sheets of paper onto which a composition without a gelling agent (i.e. comparative examples 1 and 2) is applied.

In the alternative embodiment, the composition includes:

at least 70% by weight of water, relative to the total weight of the composition, and/or

0.1 to 1% by weight of at least one gelling agent, relative to the total weight of the composition, and/or

0.1 to 10% by weight of at least one surfactant including at least one foaming surfactant, relative to the total weight of the composition.

All features and aspects disclosed in greater detail hereafter, apply to both of the aforementioned embodiments.

In a particular embodiment, the above-mentioned tissue paper is a 1-ply CWP (conventional wet-pressed)+1-ply TAD (through-air-dried) toilet paper weighing 38.5 g/m².

Once the composition is transformed into foam and applied onto the surface of 2 sheets of tissue paper placed one on top of each other, for example 2 sheets of a 1-ply CWP+1-ply TAD toilet paper weighing 38.5 g/m², the time required for said composition to soak through said sheets of paper without applied pressure, is at least two times greater than the time required for a composition without a gelling agent (i.e. comparative examples 1 and 2) to soak through said sheets of paper.

Once the composition is transformed into foam and applied onto the surface of 4 sheets of tissue paper placed one on top of each other, for example 4 sheets of a 1-ply CWP+1-ply TAD toilet paper weighing 38.5 g/m², the time required for said composition to soak through said sheets of paper with applied pressure, is at least two times greater than the time required for a composition without a gelling agent (i.e. comparative examples 1 and 2) to soak through said sheets of paper.

Once the composition is transformed into foam and applied onto the surface of 3 sheets of tissue paper placed one on top of each other, for example 3 sheets of a 1-ply CWP+1-ply TAD toilet paper weighing 38.5 g/m², the time required to tear said sheets of paper under mechanical stress, is at least two times less than the time required to tear said sheets of paper onto which a composition without a gelling agent (i.e. comparative examples 1 and 2) is applied.

In certain embodiments, the cleansing composition is for topical application, meaning an external application to the skin and/or mucous membranes. Since the composition is for topical application, it includes a physiologically acceptable medium. The term “physiologically acceptable medium” is intended to mean a medium compatible with the skin and/or mucous membranes.

In certain embodiments, the composition is foamable, meaning that when combined with a gas, such as air, it is capable of being transformed into a foam.

The composition brings effective cleanliness with small amounts of surfactant(s) and leaves a fresh and pleasant feel to the skin and improves the softness of the tissue paper on which it is applied. Furthermore, no rinsing is required with the composition.

The composition includes at least 70% by weight, or 80% by weight of water, relative to the total weight of the composition. The amount of water can range, for example, from 70% to 95% by weight, from 80% to 95% by weight, or from 85% to 95% by weight, relative to the total weight of the composition, including the stated values and all weights and weight ranges between stated values.

The composition may optionally contain, in addition to water, one or more solvents chosen from alcohols containing from 1 to 6 carbon atoms such as ethanol; polyols such as glycerol; glycols such as butylene glycol, sorbitol, isoprene glycol, propylene glycol, polyethylene glycols such as PEG-8. The amount of solvents used in the composition, in addition to water, can range from 0.5 to 20% by weight and or from 1 to 10% by weight, relative to the total weight of the composition, including the stated values and all weights and weight ranges between stated values.

The term gelling agent, also known as thickening agent, is intended to mean a polymer which swells in water and increases the viscosity of the composition, gives a better feeling, decreases the foaming properties of the composition, etc. The stability of the composition before and after being transformed into foam, is also improved by the presence of the gelling agent.

In particular embodiments, the gelling agent can in particular be chosen from:

acrylic polymers chosen from
acrylic and/or methacrylic acid homo- or co-polymers, their salts and esters such as sodium acrylate copolymer,
acrylic acid and acrylamide copolymers such as sodium polymethacrylate,
acrylates/C₁₀₋₃₀ alkyl acrylates copolymers or crosspolymers such as Pemulen® polymers, polyacrylamidomethyl propane sulfonic acid and its derivatives (partially neutralized with ammonia and highly reticulated), Carbopol® Ultrez 20 and Carbopol® Ultrez 21;
carbomers such as Carbopols® and more specifically Carbopols® Ultrez 10;
vinyl polymers chosen from polyvinylpyrrolidone (PVP), methylvinyl ether and maleic anhydride copolymers, vinylacetate and crotonic acid copolymers, vinylpyrrolidone and caprolactam copolymers, or polyvinyl alcohols;
polyethylene glycols chosen from macrogols, carbowax, monowax, hydrocire, or lutrol;
polyquaterniums such as polyquaternium 10;
polysaccharides chosen from starches such as starch from cereal grains, xanthan gum, carrageenans, agar-agar, gelose, or alginates; resins and gums chosen from Arabic gum, guar gum, tara gum, locust bean gum, or karaya gum;
cellulose derivatives selected from hydroxypropylcellulose (HPC), hydroxyethylcellulose (HEC) such as different types of Natrosol®, ethylhydroxyethyl cellulose, methylcellulose, hydroxypropyl methylcellulose, carboxymethyl cellulose, or quaternized derivatives of cellulose;
anionic, cationic, amphoteric or non-ionic chitin or chitosan polymers;
silica chosen from hydrophilic, fumed or silylated silica, such as Aerosils® including Aerosil®130, Aerosil® R812;
clays and silicates chosen from montmorillonites, hectorites, bentonites, beidellites, saponites, vermiculites, stevensites or chlorites;
   and mixtures thereof.

Clays such as montmorillonites, hectorites, bentonites, beidellites and saponites, are modified by chemical compounds selected from quaternary/tertiary amines, aminoacetates, imidazolines, amine soaps, fatty sulfates, alkyl aryl sulfonates, amine oxides, and mixtures thereof, in order to make them capable of swelling in oily media.

In a certain embodiment, the gelling agent is chosen from:

acrylic polymers chosen from acrylic and/or methacrylic acid homo- or co-polymers, their salts and esters, acrylic acid and acrylamide copolymers, acrylates/C₁₀₋₃₀alkyl acrylates copolymers, polyacrylamidomethyl propane sulfonic acid and its derivatives, carbomers, in more certain embodiments, carbomers,

cellulose derivatives selected from hydroxypropylcellulose, hydroxyethylcellulose, ethylhydroxyethyl cellulose, methylcellulose, hydroxypropyl methylcellulose, carboxymethyl cellulose, or quaternized derivatives of cellulose, in more certain embodiments, hydroxythylcellulose (HEC); and mixtures thereof.

In a still more certain embodiment, the gelling agent(s) is (are) chosen from carbomers, hydroxythylcellulose (HEC), and mixtures thereof.

When a composition is foamable, it is usually difficult to thicken the foaming medium while at the same time conserving the required properties, i.e. ability to foam in particular with a foam dispenser; good foam stability and reduced “soak through paper” properties when applied on tissue paper, in particular toilet paper. Hence, the choice of the gelling agent and the amount used in the composition of the invention is an important parameter. A cleansing composition including less than 0.1% by weight of gelling agent shows a too low “soak through properties” and foam stability. A cleansing composition including more than 1% by weight of gelling agent shows a too high viscosity and thus difficulty in transforming the composition into foam, in particular with a foam dispenser.

In a particular embodiment, the total amount of gelling agent(s) in the composition is from 0.2 to 0.5% by weight, or from 0.25 to 0.45%, by weight, relative to the total weight of the composition, including the stated values and all weights and weight ranges between stated values. The “total amount” is intended to mean the amount of all gelling agents(s) present in the composition.

As already indicated, the composition includes at least one surfactant including at least one foaming surfactant. This means that the composition may either contain only one or more foaming surfactant(s), or one or more other type(s) of surfactant(s) in addition to the foaming surfactant(s).

The term “foaming surfactant” is intended to mean a surfactant which has foaming properties when it is introduced in water.

The foaming surfactant(s) may be chosen from, but not limited to, the following surfactants:

amphoteric surfactants chosen from
betaine derivatives chosen from alkylbetaines such as cocobetaine, alkylaminobetaines (alkyl amidobetaines) such as cocamidopropylbetaine,
sultaines such as hydroxysultaine,
alkyl polyaminocarboxylates such as cocoyl polyaminocarboxylate,
alkyl amphoacetates such as disodium cocamphodiacetate, or
imidazole derivatives;

non-ionic surfactants chosen from
polycondensates of oxyethylene/oxypropylene chosen from glycerol polyoxyethylene and/or polyoxypropylene ether, fatty alcohol oxyethylene and/or oxypropylene ether such as ceteareths and more specifically ceteareth-30, fatty acid esters of glycerol oxyethylene and/or oxypropylene such as PEG-200 glyceryl monostearate, fatty acid esters of sorbitol polyoxyethylene and/or polyoxypropylene such as polysorbates and more specifically polysorbate-60, oxyethylene and oxypropylene copolymers, ethylene and propylene oxide condensates with fatty alcohols, polyethylene glycol fatty acid esters such as PEG-50 stearate, alkylpolyglucoside optionally oxyalkylated, alkylglucoside esters such as decyl glucoside, fatty acid esters of sorbitan optionally oxyethylated, polyoxyalkylated fatty acid esters,
amine derivatives chosen from N-alkylglucamine and N-acylméthylglucamine derivatives, amines, amine oxides, polyethoxylated or polyglycerolated fatty amines, polyglycerolated diglycolamides,
silicone derivatives chosen from dimethicone copolyol, dimethicone copolyol benzoate, cyclomethicone/dimethicone copolyol,
saccharide derivatives chosen from esters and ethers of monosaccharide such as sucrose stearate, fatty acid esters of saccharose such as sucrose stearate,
ethylene oxide derivatives chosen from alkoxylated sugar esters such as PEG-120 methylglucose dioleate;
   and mixtures thereof.

In a particular embodiment, the composition includes at least one foaming surfactant, said foaming surfactant(s) being a betaine derivative chosen from alkylbetaines such as cocobetaine, alkylaminobetaines (alkyl amidobetaines) such as cocamidopropylbetaine, and mixtures thereof.

The total weight of foaming surfactant(s) in the composition ranges from 0.1 to 10% by weight, or from 0.5 to 6% by weight, relative to the total weight of the composition, including the stated values and all weights and weight ranges between stated values. The “total weight” in this embodiment is intended to mean the weight of all foaming surfactants present in the composition.

As previously mentioned, the composition may only contain foaming surfactant(s). The composition may further contain or one or more other surfactant(s) in addition to the foaming surfactant(s). The total weight of surfactant(s) including the foaming surfactant(s) in the composition, ranges from 0.1 to 10% by weight, or from 0.5 to 6% by weight, relative to the total weight of the composition, including the stated values and all weights and weight ranges between stated values. The “total weight” is intended to mean the weight of all surfactants present in the composition.

The additional surfactants are mainly intended to lower the surface tension of water and other liquids or that between a liquid and a solid. They may thus act, for example, as detergents, wetting agents, emulsifiers and dispersants. They may also have foaming properties.

The additional surfactant(s) may be chosen from:

anionic surfactants chosen from
anionic derivatives of proteins (apple, wheat, soya, oat, silk protein) and polypeptide derivatives;
carboxylates chosen from amido ether carboxylates such as sodium lauryl amido ether carboxylate, polyoxthyethylene carboxylates such as sodium lauryl ether carboxylate, fatty acid salts having an C₁₆₋₂₂ alkyl chain neutralized by an organic or inorganic base, which constitute soaps (alkaline soaps amine soaps of formula RCOOM with M being Na, K, NH₄⁺ or triethanolamine and R═C₁₆₋₂₂ alkyl chain);
aminoacid derivatives chosen from alkylsarcosinates such as sodium lauroyl sarcosinate (C₁₂H₂₅—CON(CH₃)—CH₂—COONa), alalinates such as sodium N-lauroyl N-methyl amido propionate, glutamates such as triethanolamine cocoyl glutamate, aspartates such as ethanolamine N-lauroyl aspartate or ethanolamine N-myristoyl aspartate, glycinates such as sodium N-cocyl glycinate, citrates such as citric monoester of coco alcohol ethoxylate;
tartrates such as sodium salt of palm oil methyltaurate;
sulfate derivatives chosen from sodium, ammonium, potassium, triethanolamine alkyl sulfates such as sodium lauryl sulfate; sodium, ammonium, potassium, triethanolamine alkyl ether sulfates such as sodium lauryl ether sulfate;
sulfonate derivatives chosen from paraffins and olein solfonates such as sodium alpha olefin sulfonate, alkyl sulfoacetates (R—SO₃—CH₂—COOM), isethionates (R—COO—CH₂—CH₂—SO₃Na) such as sodium cocoylisethionate;
alkyl polyglucoside anionic derivatives chosen from sulfosuccinates, citrates, tartrates, carbonates and ethers of glycerol obtained from alkylglucosides such as mono-sulfosuccinate of lauric alcohol; or
phosphated derivatives chosen from sodium alkyl phosphates (RO—PO₃—Na₂) or alkyl ether phosphates de sodium R—(C₂H₄O)ₙPO₃Na₂;

cationic surfactants chosen from quaternary ammoniums, alkylpyridinium chlorides, alkylammonium saccharinates, or aminoxydes having a pH<6.5;

and mixtures thereof.

The composition may contain further additives and/or active agents chosen from, for example:
preservatives or preserving agents, non-limitative examples thereof being chlorhexidine, sodium chlorate; chlorphenesin; phenoxyethanol such as Phenoxetol®; a mixture of phenoxyethanol, methylparaben and ethylparaben such as Phenonip®; a mixture of phenoxyethanol, methylparaben and propylparaben such as Nipaguard BPX®; PEG-4 laurate such as Nipaguard IPF®; iodopropynyl butylcarbamate; a mixture of triethylene glycol, benzyl alcohol and propylene glycol such as Nipaguard CMB®; sodium hydroxymethylglycinate such as Nipaguard SMG®, sorbic acid, dehydroacetic acid, sodium benzoate, potassium sorbate, lyaminopropyl biguanide (PAPB), benzoic acid;
sequestering or chelating agents such as ethylenediaminetetraacetic acid (EDTA), ethylenediamine tetra(methylene phosphonic acid) (EDTMP), diethylenetriamine penta(methylene phosphonic acid) (DTPMP), nitrilotriacetic acid (NTA), pentetic acid or diethylene triamine pentaacetic acid (DTPA);
antioxidants;
fragrances such as eucalyptus oil, clove oil, jojoba oil, lavender oil; fleur d'eau MIP 1101016 from Expressions Parfumées;
pH modifiers such as sodium hydroxide, citric acid;
antiseptics; antimicrobials such as benzoyl peroxide, salicylic acid, triclosan;
natural extracts having for example soothing, moisturizing, and healing properties, such as aloe vera;
soothing agents or emollients such as allantoin; panthenol; polyols such as glycerol or glycerin, sorbitol; glycols such as butylene glycol, isoprene glycol; polyethylene glycols such as PEG-8, methoxypolyethylene glycols;

and mixtures thereof.

The amounts of these various additives are those conventionally used in this field, and complete the weight of the composition to a total weight of 100%. These additives and the concentrations thereof should be such that they do not modify the property desired for the composition and that they do not destabilize it.

In a particular embodiment, the composition contains at least a soothing agent or emollient. The soothing agents or emollients can be chosen from panthenol; polyols such as glycerol or glycerin; glycols such as butylene glycol, and mixtures thereof.

The amount of emollients in the composition ranges, for example, from 0.1 to 5% by weight relative to the total weight of the composition, including the stated values and all weights and weight ranges between stated values.

According to a particular embodiment, the composition includes:

88 to 92% by weight of water,

0.25 to 0.45% by weight of a gelling such as carbomer and/or hydroxyethylcellulose,

1.5 to 2.5% by weight of a foaming surfactant such as cocamidopropylbetaine,

0.2 to 2.5% by weight of emollients such as butylene glycol and/or panthenol,

0.1 to 1% by weight of preservatives such as caprylyl glycol and/or phenoxyethanol,

0.5 to 1.5% by weight of a natural extract such as aloe vera,

0.15 to 2.7% by weight of a pH modifier such as sodium hydroxide or citric acid,

0.1 to 0.15% by weight of a chelating agent such as EDTA,

0.1 to 0.2% by weight of a fragrance such as Fleur d'ea MIP 1101016,

all weights being relative to the total weight of the composition.

In certain embodiments, the composition is in the form of a gel.

In certain embodiments, the composition has a viscosity ranging, for example, from 0.001 to 1 Pa·s, measured at a temperature of 25° C., using a Brookfield LV DV I+ from BROOKFIELD ENGINEERING LABORATORIES, INC. In particular embodiments, the viscosity of the composition ranges from 0.001 to 0.500 Pa·s, or from 0.010 to 0.350 Pa·s. The viscosity measuring device is equipped with a set of four spindles: LV1, LV2, LV3 and LV4. The following spindles (sp) and rotational speeds (v) are used:

| | |
|---|---|
| sp 1/v 100 rpm | for η < 0.1 Pa · s (100 cP) |
| sp 2/v 50 rpm | for 0.1 Pa · s (100 cP) < η < 1 Pa · s (1000 cP) |
| sp 2/v 10 rpm | for 1 Pa · s (1000 cP) < η < 3 Pa · s (3000 cP) |
| sp 3/v 50/20/10 rpm | for 3 Pa · s (3000 cP) < η < 6 Pa · s (6000 cP). |

The composition is foamable meaning that it is specifically formulated to form a foam when aerated. Said foam may be formed by using, for example, a foam dispenser.

In particular embodiments, the foam dispenser is easy to use, forms foam instantly, in a precise dosage, allows an improved spread of effective ingredients, and without use of propellants.

The composition may be contained in a dispensing foam pump container using, for example, a positive displacement pump that acts directly on the composition. The pump draws the composition up a siphon tube from the bottom of the container, and the composition is forced out a nozzle.

For example, the dispensing foam pump container may be an aerosol container. In an aerosol container, the composition is maintained under pressure sufficient to cause foam formation when dispensed. Of particular advantage, however, the composition is foamable without necessity of being placed in an aerosol container.

Airspray pump foamers allowing precise mixing of the composition and air without using gas propellants, can be used. Their use results in a high quality foam with each single stroke.

Foam dispensers may be chosen from F2-L11, F3-L11, G3-L11, WRT4-L11, WRT-L11, F2-L9, F3-L9, G3-L9, T1-L9, WRT4-L9, WRT-L9, F2-L7, F3-L7, G3-L7, T1-L7, WRT4-L7, or WRT-L7 sold by Rexam. Other foam dispensers supplied by other suppliers may also be used.

Automatic touchless foam dispensers may also be used. An example of such automatic touchless foam dispenser is: enMotion® Automated Touchless Soap Dispenser.

Said foamer dispensers entrain air in the composition as it is dispensed.

Also described herein is the use of the composition for cleansing of the skin and/or mucous membrane.

Additionally, the composition can be used with tissue paper, in particular toilet paper, for cleansing of the skin and/or mucous membrane.

Also described herein is a method for cleansing skin and/or mucous membrane including:

(i) applying the composition to a tissue paper, in particular toilet paper, and
(ii) cleansing the areas in need of being cleansed.

In step (i), when applied onto the tissue paper, the composition is in the form of a foam. The cleansing step (ii) may be carried out by wiping the areas to be cleansed with the tissue paper including said foamed composition on its surface.

The present disclosure also relates to a method for cleansing skin and/or mucous membrane wherein the composition is

(i) applied directly on the areas to be cleansed, and
(ii) cleansing said areas by wiping with a tissue paper, in particular toilet paper.

The cleaning composition may be applied onto all types of tissue papers such as hankies, facial tissues, tissue wipes, hand towels, kitchen towels, for cleaning any type of supports, and in particular for cleansing the skin or the mucous membranes. In a particular embodiment, the tissue paper is toilet paper. In a more particular embodiment, the tissue paper, in particular toilet paper, does not contain a wet strength agent.

The composition may also be applied directly on the skin and/or mucous membrane to be cleansed, and then rinsed off with water. However, this is not a preferred embodiment of the present invention.

The present disclosure further concerns a kit for cleansing of the skin and/or mucous membranes including the composition and a dispensing foam pump container. Said kit may further include tissue paper, in particular toilet paper.

The present disclosure also concerns a container provided with a dispensing foam pump and containing a composition.

The present disclosure further concerns an automatic foam dispenser containing a composition according to an embodiment of the invention.

The present disclosure also concerns the use of a composition according to an embodiment of the invention with tissue paper, in particular toilet paper, for cleansing of the skin and/or mucous membrane.

The present disclosure further concerns a tissue paper product, in particular a toilet paper product, including a composition including:
at least 70% by weight of water, relative to the total weight of the composition;
0.1 to 1% by weight of at least one gelling agent, relative to the total weight of the composition, and
0.1 to 10% by weight of at least one surfactant including at least one foaming surfactant, relative to the total weight of the composition.

The present disclosure also concerns a tissue paper product, in particular a toilet paper product, including a composition according to an embodiment of the invention.

### BRIEF DESCRIPTION OF THE FIGURES

Other advantages and features of the present invention may be better understood with respect to the following examples given for illustrative purposes and the accompanying figures:

FIGS. 1 and 2 represent the equipment for measuring the “soak through properties” of different compositions including the composition of the invention, i.e. the time required for a composition to soak through toilet paper without applied pressure. A detailed description of the equipment in FIGS. 1 and 2 is provided in the examples, i.e. Method 1;

FIG. 3 represents the equipment for measuring the stability of the foam for different compositions. A detailed description of the equipment used is provided in the examples, i.e. Method 3;

FIG. 4 represents the stability of the foam (the amount of foam transformed back into liquid over time) measured for different compositions for compositions: no. 3 and Aaah® without a gelling agent (comparative examples 1 and 2), and compositions nos. 56 to 59 according to embodiments of the invention containing a gelling agent (0.45% and 0.25% by weight of Carbopol Ultrez 10 respectively for compositions nos. 56 and 57; and 0.45% and 0.25% by weight of Natrosol HHX respectively for compositions nos. 58 and 59);

FIG. 5 represents the equipment used for determining the strength of the toilet paper onto which different foamed compositions are applied. A detailed description of the equipment used is provided in the examples, i.e. Method 4.

### DETAILED DESCRIPTION OF PREFERRED EMBODIMENTS

### Examples

### Examples 1 to 4

### Compositions According to Embodiments of the Invention

Compositions nos. 56 to 59 according to embodiments of the invention were prepared in a conventional manner, for example, by simple admixture of components at room temperature (20° C.+5° C.), and tested.

The amount of each component of the composition is given in % by weight relative to the total weight of the composition. The pH of the different compositions is 5.7+0.2. Products no. 56 to 59 are compositions according to embodiments of the invention including a gelling agent.

Phase A

| | Commercial | | Product | Product | Product | Product |
|---|---|---|---|---|---|---|
| INCI name | name/Supplier | Function | no. 56 | no. 57 | no. 58 | no. 59 |
| Water (Aqua) | Purified water/ETC | Solvent | 88.35% | 90.05% | 90.90% | 91.05% |
| Butylene | 1,3-Butanediol/ | Emollient | 2.00% | 2.00% | 2.00% | 2.00% |
| Glycol | Sigma Aldrich | | | | | |
| Panthenol | D-Panthenol | Emollient | 0.20% | 0.20% | 0.20% | 0.20% |
| | USP/BASF | | | | | |
| Tetrasodium | EDTA 4Na/Sigma | Chelating | 0.10% | 0.10% | 0.10% | 0.10% |
| EDTA | Aldrich | agent | | | | |
| Aloe | Aloe Vera Gel 10X | Active | 1.00% | 1.00% | 1.00% | 1.00% |
| Barbadensis | AG014P/Rahn | ingredient | | | | |
| Leaf Juice | | | | | | |
| Carbomer | Carbopol Ultrez | Gelling | 0.45% | 0.25% | — | — |
| | 107 Noveon - | agent | | | | |
| | Gattefossé | | | | | |
| Hydroxyethyl | Natrosol HHX/ | | — | — | 0.45% | 0.25% |
| cellulose | Hercules/Ashland | | | | | |
| | | | 92.10% | 93.60% | 94.65% | 94.60% |

Phase B

| | Commercial | | Product | Product | Product | Product |
|---|---|---|---|---|---|---|
| INCI name | name/Supplier | Function | no. 56 | no. 57 | no. 58 | no. 59 |
| Cocamido- | Dehyton PK | Foaming | 4.00% | 4.00% | 4.00% | 4.00% |
| propyl | 45/Cognis | surfactant | (1.80% AM)* | (1.80% AM)* | (1.80% AM)* | (1.80% AM)* |
| betaine | | | | | | |
| Perfume | Fleur d'eau MIP | Perfume | 0.20% | 0.20% | 0.20% | 0.20% |
| | 1101016/Expressions | | | | | |
| | Parfumées | | | | | |
| Caprylyl | Dermosoft Octiol/ | Preservative | 0.10% | 0.10% | 0.10% | 0.10% |
| Glycol | Dr Straetmans | | | | | |
| | (Lucas Meyer | | | | | |
| Phenoxy- | Phenoxetol/SCD- | Preservative | 0.90% | 0.90% | 0.90% | 0.90% |
| ethanol | Clariant | | | | | |
| | | | 5.20% | 5.20% | 5.20% | 5.20% |

*amount expressed on an active material basis: the active material is the INCI component(s) being the surfactant(s) comprised in the commercial composition

Phase C

| | Commercial | | Product | Product | Product | Product |
|---|---|---|---|---|---|---|
| INCI name | name/Supplier | Function | no. 56 | no. 57 | no. 58 | no. 59 |
| Sodium | Sol. NaOH 1N/ | pH modifier | 2.70% | 1.20% | — | — |
| hydroxide | Sigma Aldrich | | | | | |
| | (sol. ETC) | | | | | |
| Citric Acid | Sol. Acide 10%/ | Solvent/pH | — | — | 0.15% | 0.20% |
| | Sigma Aldrich | modifier | | | | |
| | (sol. ETC) | | | | | |
| | | | 2.700% | 1.200% | 0.150% | 0.200% |

The viscosity of each composition is measured at a temperature of 20 to 25° C., using a Brookfield LV DV I+ from BROOKFIELD ENGINEERING LABORATORIES, INC, as indicated previously. The results are summarized in the table below.

| | Product | Product | Product | Product |
|---|---|---|---|---|
| | no. 56 | no. 57 | no. 58 | no. 59 |
| Viscosity | 0.220 Pa · s | 0.010 Pa · s | 0.18 Pa · s | 0.02 Pa · s |
| (Brookfield LV | (220 cP) | (10 cP) | (180 cP) | (20 cP) |
| DV I+) | (sp2, v50) | (sp2, v100) | (sp2, v50) | (sp1, v100) |

### Comparative Example 1

### Composition without a Gelling Agent

Comparative composition no. 3 was prepared according to the same experimental procedure as for composition no. 58 in Examples 1 to 4.

The only difference between composition no. 3 and composition no. 58 is the absence of a gelling agent and the amount of water.

Phase A

| | | | Product |
|---|---|---|---|
| INCI name | Commercial name/Supplier | Function | no. 03 |
| Water (Aqua) | Purified water/ETC | Solvent | 91.35% |
| Butylene | 1,3-Butanediol/Sigma Aldrich | Emollient | 2.00% |
| Glycol | | | |
| Panthenol | D-Panthenol USP/BASF | Emollient | 0.20% |
| Tetrasodium | EDTA 4Na/Sigma Aldrich | Chelating | 0.10% |
| EDTA | | agent | |
| Aloe | Aloe Vera Gel 10X AG014P/Rahn | Active | 1.00% |
| Barbadensis | | ingredient | |
| Leaf Juice | | | |
| | | | 94.65% |

Phase B

| | | | Product |
|---|---|---|---|
| INCI name | Commercial name/Supplier | Function | no. 03 |
| Cocamido- | Dehyton PK 45/Cognis | Foaming | 4.00% |
| propyl | | surfactant | (1.80% AM)* |
| betaine | | | |
| Perfume | Fleur d'eau MIP 1101016/ | Perfume | 0.20% |
| | Expressions Parfumées | | |
| Caprylyl | Dermosoft Octiol/ | Preservative | 0.10% |
| Glycol | Dr Straetmans | | |
| | (Lucas Meyer | | |
| Phenoxy- | Phenoxetol/SCD-Clariant | Preservative | 0.90% |
| ethanol | | | |
| | | | 5.20% |

*amount expressed on an active material basis: the active material is the INCI component(s) being the surfactant(s) comprised in the commercial composition

Phase C

| | | | Product |
|---|---|---|---|
| INCI name | Commercial name/Supplier | Function | no. 03 |
| Sodium | Sol. NaOH 1N/Sigma Aldrich | pH modifier | — |
| hydroxide | (sol. ETC) | | |
| Citric | Sol. Acide 10%/Sigma | Solvent/pH | 0.15% |
| Acid | Aldrich (sol. ETC) | modifier | |
| | | | 0.15% |

Composition no. 3 is a liquid.

### Comparative Example 2

### Composition Aaah®, commercialized by Aaah Company

Commercial composition Aaah®, sold by The Aaah Company LLC 6590 S Vine Street #112 Centennial, CO 80121-2762 has the following composition:

| INCI name | Function |
|---|---|
| Water (Aqua) | Solvent |
| Cocamidopropyl betaine | Foaming surfactant |
| Glycerin | Emollient |
| Sodium chlorate | Deodorant |
| Whole leaf aloe vera concentrate | Natural extract |
| Commiphora Gileadenis (balm of | Natural extract |
| Gilead) extract | |
| Allantoin | Soothing agent |

Aaah® composition differs from compositions nos. 56 to 59 essentially by the absence of gelling agent, pH modifier, chelating agent and perfume.

Products nos. 56 to 59 (according to embodiments of the invention) were tested and compared to product no. 3 and commercialized Aaah® product.

Each composition was transformed into a foam by a foaming pump dispenser, reference F2-L11 manufactured by the company Rexam, and applied on sheets of toilet paper.

Several toilet papers were tested:

### 2-Ply Toilet Paper:

Lotus Confort: 1-ply CWP+1-ply TAD (100% virgin fibers); weight 38.5 g/m²

Auchan 2-ply; 2-ply CWP (100% virgin fibers); weight 38.5 g/m²

Lotus Professional Next Turn Compact 2-ply CWP (100% recycled fibers); weight 30 g/m²

### 3-Ply Toilet Paper:

Moltonel; 3-ply CWP (100% virgin fibers); weight 54 g/m²
Several methods have been used to compare the effectiveness of different foams.
Method 1: Measure of the Time Required for the Composition to Soak Through Toilet Paper without Applied Pressure

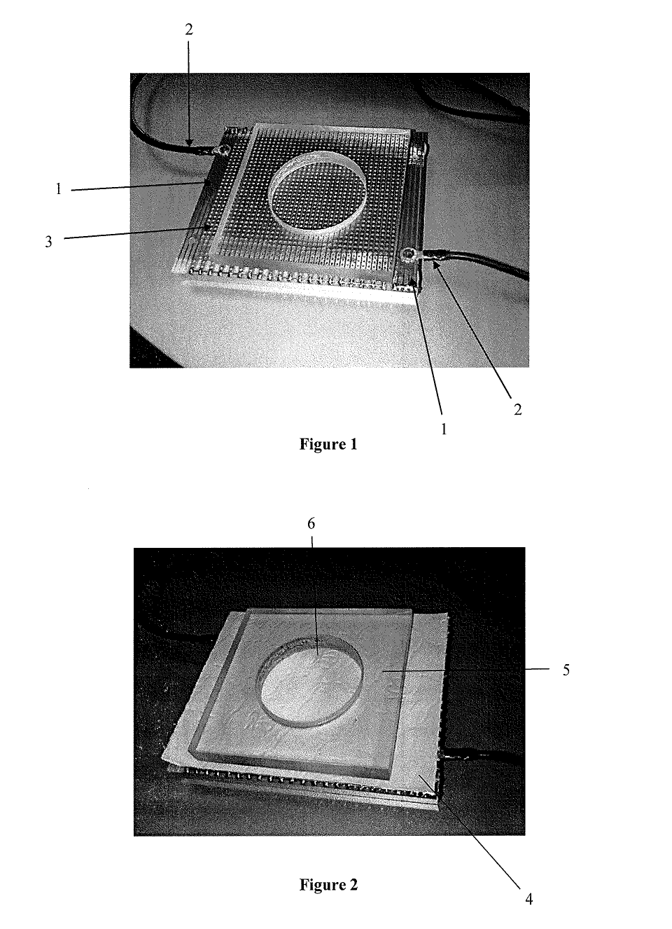

The sensor shown in FIG. 1 is used to measure the time required for a liquid composition to soak through the toilet paper. The sensor consists of a printed circuit (1). This circuit has two terminals (2), each of which is connected to a series of conductive wires (3). The wires are intertwined so that each wire of a terminal is positioned between the two wires of the other terminal. All of the wires are parallel to each other, have a width of 1.8 mm and are spaced 0.7 mm. The terminals (2) are connected to a lamp (not represented). When a conductive liquid is poured onto the printed circuit (1), the electrical current is conducted by the liquid between the two terminals (2) and lights the lamp.

Several sheets (i.e. 2 or 6 sheets) of toilet paper (4) were placed on the printed circuit and maintained by a plastic plate (5) with a hole (6) so that they remain flat against the printed circuit. This is shown in FIG. 2. A dose of foam is sprayed onto the top sheet at a distance of between 30 and 60 mm, with an angle of between 45 and 90° from the vertical. A dose of foam equals to 0.75 g of the composition. At the same time a chronometer is started. As soon as the lamp (not represented) is lit, the chronometer is stopped and the time required for the fluid to pass through the two sheets (4) is noted.

The method is repeated identically for each product and each toilet paper. The following results are obtained (average of 5 measurements).

| | | | | Time required for the liquid composition to soak through the | | | | | |
|---|---|---|---|---|---|---|---|---|---|
| | | | | toilet paper and trigger the sensor in seconds | | | | | |
| | | | | | | | | Product | Product |
| | | No. of | | | | Product | Product | no. 58 | no. 59 |
| | | sheets | No. of | Aaah ® | Product | no. 56 | no. 57 | 0.45% | 0.25% |
| | No. of | super- | doses | Product | no. 3 | 0.45% | 0.25% | Hydroxyethyl- | Hydroxyethyl- |
| | plies | posed | of foam | — | — | Carbomer | Carbomer | cellulose | cellulose |
| Lotus | 2 | 2 | 1 | 1.4 | 1.3 | 132 | 21 | 28 | 4 |
| Confort | | | | | | | | | |
| Auchan | 2 | 2 | 1 | 0.8 | 0.7 | 80 | 13 | 17 | 1.7 |
| Pure | | | | | | | | | |
| Douceur | | | | | | | | | |
| Lotus | 2 | 6 | 1 | 1.0 | 1.0 | >240 | 199 | 14 | 2.3 |
| Professional | | | | | | | | | |
| Moltonel | 3 | 2 | 1 | 2.4 | 2.4 | 319 | 85 | 79 | 5.0 |

These results show that the time required for the compositions according to embodiments of the invention, which contain a gelling agent, to soak through two superposed sheets of the different toilet papers used, is more than two times greater than compositions no. 3 and Aaah® which do not contain any gelling agent. The improved “soak though” property (soak through speed is reduced) of the compositions according to embodiments of the invention is thus due to the presence of a gelling agent.

Method 2: Measure of the Time Required for the Composition to Soak Through Toilet Paper with Applied Pressure

A pile of toilet paper is prepared by superposing several sheets (i.e. 4, 6 or 8 sheets) one on another. This pile is placed on the printed circuit. A dose of foam is sprayed onto the top sheet at a distance of between 30 and 60 mm, with an angle of between 45 and 90° from the vertical. At the same time, a chronometer is started. A plastic plate weighing 194 g with a surface area of 81 cm² is placed on the foam as quickly as possible. As soon as the lamp is lit, the chronometer is stopped and the time required for the fluid to soak through the four sheets is noted.

The following results are obtained (average of 5 measurements).

| | | | | Time required for the liquid composition to soak | | | | | |
|---|---|---|---|---|---|---|---|---|---|
| | | | | through the toilet paper and trigger the sensor in seconds | | | | | |
| | | No. of | | | | Product | Product | Product | Product |
| | | sheets | No. of | Aaah ® | Product | no. 56 | no. 57 | no. 58 | no. 59 |
| | No. of | super- | doses | Product | no. 3 | 0.45% | 0.25% | 0.45% | 0.25% |
| | plies | posed | of foam | — | — | Carbomer | Carbomer | HEC | HEC |
| Lotus | 2 | 4 | 1 | 0 | 0 | 116 | 75 | 27.2 | 4 |
| Confort | | | | | | | | | |
| Auchan | 2 | 6 | 1 | 0.6 | 0.8 | >240 | >240 | 25 | 3 |
| Pure | | | | | | | | | |
| Douceur | | | | | | | | | |
| Lotus | 2 | 8 | 1 | 0.1 | 0.5 | >240 | >240 | >240 | 5 |
| Professional | | | | | | | | | |
| Moltonel | 3 | 4 | 1 | 0.8 | 1.6 | >240 | 53 | >240 | 49 |

From the results obtained, it appears clearly that the time required for the compositions according to embodiments of the invention, which contain a gelling agent, to soak through four superposed sheets of the different toilet papers used, even when pressure is applied, is more than two times greater than compositions no. 3 and Aaah® which do not contain any gelling agent. These results show the improved “soak through” property (soak through speed is reduced) of the compositions according to embodiments of the invention due to the presence of the gelling agent.

### Method 3: Measure of Foam Stability

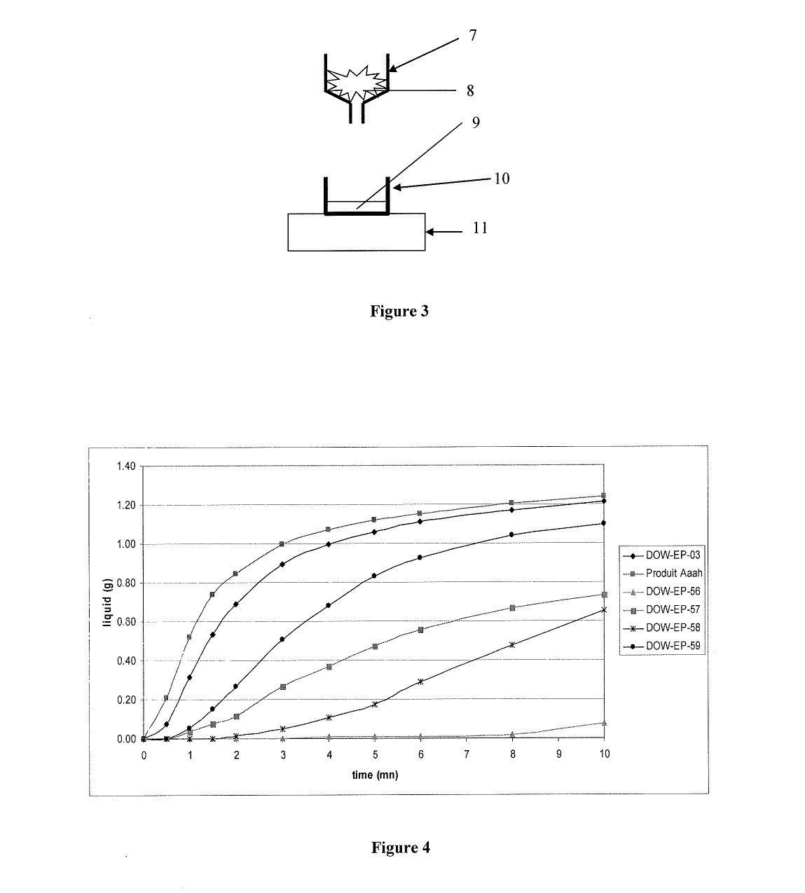

The amount of foam transformed back into liquid over time shows the stability of a composition. The stability of compositions nos. 56 to 59 (according to embodiments of the invention) was measured and compared to the stability of compositions no. 3 and Aaah®, using the equipment illustrated in FIG. 3. Two doses of foam (8) are sprayed in the funnel (7), and at the same time a chronometer is started. The weight of liquid (9) collected in recipient (10) and displayed on the scale (11) is regularly noted. The results are illustrated in FIG. 4.

Based on the results illustrated in FIG. 4, it appears that the compositions according to embodiments of the present invention are transformed into liquid at least two times less fast than the comparative compositions (no. 3 and Aaah®) which do not contain any gelling agent.

Based on these results, it is clear that the presence of a gelling agent improves the stability of the compositions.

Method 4: Measure of the Time Required for the Tissue Paper onto which the Foam is Applied to Tear Under a Mechanical Stress (Strength of the Paper)

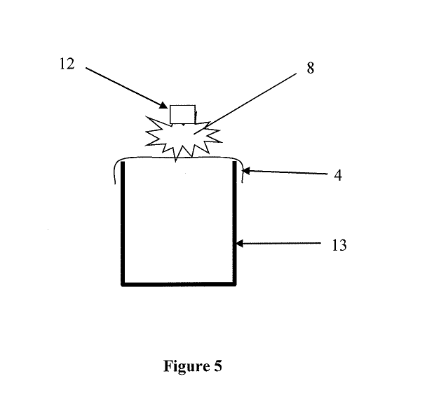

Several sheets (i.e. 2 or 3 sheets) of toilet paper (4) are fixed on a 53 mm diameter cylindrical container (13) with a rubber band (not represented) so as to have an evenly distributed voltage. A dose of foam (8) is sprayed onto the top sheet at a distance of between 30 and 60 mm, with an angle of between 45 and 90° from the vertical. A cylindrical weight (12) having a diameter of 20 mm, a height of 21.5 mm and a weight of 50 g, is placed on the sheet. This is illustrated in FIG. 5. At the same time a chronometer is started. When the sheet (4) is torn by the action of weight (12) and liquid, the chronometer is stopped and the time required by the liquid to tear the several sheets is noted.

The following results are obtained (average of 5 measurements).

| | | | | Time required for the liquid composition to soak | | | | |
|---|---|---|---|---|---|---|---|---|
| | | | | through the toilet paper and trigger the sensor in seconds | | | | |
| | | No. of | | | | Product | Product | Product |
| | | sheets | No. of | Aaah ® | Product | no. 56 | no. 57 | no. 58 |
| | No. of | super- | doses | Product | no. 3 | 0.45% | 0.25% | 0.45% |
| | plies | posed | of foam | — | — | Carbomer | Carbomer | HEC |
| Lotus | 2 | 3 | 1 | 8 | 4 | >400 | >400 | 44 |
| Confort | | | | | | | | |
| Auchan | 2 | 3 | 1 | 1 | 1 | 50 | 10 | 16 |
| Pure | | | | | | | | |
| Douceur | | | | | | | | |
| Lotus | 2 | 3 | 1 | 1 | 1 | 153 | 19 | 3 |
| Professional | | | | | | | | |
| Moltonel | 3 | 2 | 1 | 10 | 11 | 119 | 43 | 15 |

It appears from these results that under mechanical stress, the time required for superposed sheets of the toilet paper onto which the foam is applied, to tear, is more than two times less than the time required for superposed sheets of the toilet paper onto which comparative compositions (no. 3 and Aaah®) without a gelling agent are applied, to tear.

### Examples 5 and 6

### Compositions According to Embodiments of the Invention

Compositions nos. 55 and 60 according to embodiments of the invention were prepared in a conventional manner, for example, by simple admixture of components at room temperature (20° C.+5° C.), and tested.

The amount of each component of the composition is given in % by weight relative to the total weight of the composition. The pH of the different compositions is 5.7+0.2.

Phase A

| INCI | Commercial | | Product | Product |
|---|---|---|---|---|
| name | name/Supplier | Function | no. 55 | no. 60 |
| Water | Purified | Solvent | 88.30% | 90.10% |
| (Aqua) | water/ETC | | | |
| Butylene | 1,3-Butanediol/ | Emollient | 2.00% | 2.00% |
| Glycol | Sigma Aldrich | | | |
| Panthenol | D-Panthenol | Emollient | 0.20% | 0.20% |
| | USP/BASF | | | |
| Tetrasodium | EDTA 4Na/ | Chelating | 0.10% | 0.10% |
| EDTA | Sigma Aldrich | agent | | |
| Aloe | Aloe Vera Gel | Active | 1.00% | 1.00% |
| Barbadensis | 10X | ingredient | | |
| Leaf Juice | AG014P/Rahn | | | |
| Carbomer | Carbopol | Gelling | 0.5% | 0.20% |
| | Ultrez 10/ | agent | | |
| | Noveon - | | | |
| | Gattefossé | | | |
| | | | 92.10% | 93.60% |

Phase B

| INCI | Commercial | | Product | Product |
|---|---|---|---|---|
| name | name/Supplier | Function | no. 55 | no. 60 |
| Cocamido- | Dehyton PK | Foaming | 4.00% | 4.00% |
| propyl | 45/Cognis | surfactant | (1.80% AM)* | |
| betaine | | | | |
| Perfume | Fleur d'eau MIP | Perfume | 0.20% | 0.20% |
| | 1101016/Expres- | | | |
| | sions Parfumées | | | |
| Caprylyl | Dermosoft Octiol/ | Preser- | 0.10% | 0.10% |
| Glycol | Dr Straetmans | vative | | |
| | (Lucas Meyer | | | |
| Phenoxy- | Phenoxetol/SCD- | Preser- | 0.90% | 0.90% |
| ethanol | Clariant | vative | | |
| | | | 5.20% | 5.20% |

*amount expressed on an active material basis: the active material is the INCI component(s) being the surfactant(s) comprised in the commercial composition

Phase C

| INCI | Commercial | | Product | Product |
|---|---|---|---|---|
| name | name/Supplier | Function | no. 55 | no. 60 |
| Sodium | Sol. NaOH 1N/Sigma | pH modifier | 2.70% | 1.20% |
| hydroxide | Aldrich (sol. ETC) | | | |
| | | | 2.700% | 1.200% |

The viscosity of each composition is measured at a temperature of 25° C., using a Brookfield LV DV I+ from BROOKFIELD ENGINEERING LABORATORIES, INC, as indicated previously.

Composition no. 55 showed a higher viscosity than composition no. 56, which may render the pumping slightly more difficult. The stability of this composition is lower than that of composition no. 56 but higher than the stability of comparative examples 1 and 2.

Composition no. 60 showed a lower viscosity than composition no. 57. Its soak through properties are not as good as composition no. 57 but still remain satisfactory.

1. A composition comprising:
at least 70% by weight of water, relative to the total weight of the composition;
0.1 to 1% by weight of at least one gelling agent, relative to the total weight of the composition, and
0.1 to 10% by weight of at least one surfactant including at least one foaming surfactant, relative to the total weight of the composition.

2. The composition according to claim 1, wherein said composition satisfies at least one of the following conditions:
once transformed into foam and applied onto the surface of several sheets of tissue paper placed one on top of each other, the time required for said composition to soak through said sheets of paper, with or without applied pressure, is at least two times greater than the time required for a composition without a gelling agent to soak through said sheets of paper, and/or
the foam stability of said composition is such that after two minutes, the said composition is transformed into liquid at least two times less fast than a composition without a gelling agent, and/or
once transformed into foam and applied onto the surface of several sheets of tissue paper, the time required to tear said sheets of paper under a mechanical stress, is at least two times less than the time required to tear said sheets of paper onto which a composition without a gelling agent is applied.

3. A composition comprising water, at least one gelling agent and at least one surfactant including at least one foaming surfactant, wherein said composition satisfies at least one of the following conditions:
once transformed into foam and applied onto the surface of several sheets of tissue paper placed one on top of each other, the time required for said composition to soak through said sheets of paper, with or without applied pressure, is at least two times greater than the time required for a composition without a gelling agent to soak through said sheets of paper, and/or
the foam stability of said composition is such that after two minutes, the said composition is transformed into liquid at least two times less faster fast than a composition without a gelling agent, and/or
once transformed into foam and applied onto the surface of several sheets of tissue paper, the time required to tear said sheets of paper under a mechanical stress, is at least two times less than the time required to tear said sheets of tissue paper onto which a composition without a gelling agent is applied.

4. The composition according to claim 3, comprising
at least 70% by weight of water, relative to the total weight of the composition, and/or
0.1 to 1% by weight of at least one gelling agent, relative to the total weight of the composition, and/or
0.1 to 10% by weight of at least one surfactant including at least one foaming surfactant, relative to the total weight of the composition.

5. The composition according to claim 2, wherein once transformed into foam and applied onto the surface of 2 sheets of tissue paper placed one on top of each other, the time required for said composition to soak through said sheets of paper without applied pressure, is at least two times greater than the time required for a composition without a gelling agent to soak through said sheets of paper.

6. The composition according to claim 2, wherein once transformed into foam and applied onto the surface of 4 sheets of tissue paper placed one on top of each other, the time required for said composition to soak through said sheets of paper with applied pressure, is at least two times greater than the time required for a composition without a gelling agent to soak through said sheets of paper.

7. The composition according to claim 2, wherein once transformed into foam and applied onto the surface of 3 sheets of tissue paper placed one on top of each other, the time required to tear said sheets of paper under a mechanical stress, is at least two times less than the time required to tear said sheets of paper onto which a composition without a gelling agent is applied.

8. The composition according to claim 2, wherein the tissue paper is a 1-ply CWP+1-ply TAD toilet paper weighing 38.5 g/m².

9. The composition according to claim 1, wherein said composition has a viscosity ranging from 0.001 to 0.500 Pa·s.

10. The composition according to claim 1, wherein the amount of water ranges from 70% to 95% by weight, relative to the total weight of the composition.

11. The composition according to claim 1, wherein the gelling agent is chosen from the group consisting of acrylic polymers; vinyl polymers; polyethylene glycols; polyquaterniums; polysaccharides; cellulose derivatives; anionic, cationic, amphoteric or non-ionic chitin or chitosan polymers; silica; clays and silicates; and mixtures thereof.

12. The composition according to claim 1, wherein the gelling agent is chosen from the group consisting of:
acrylic polymers chosen from the group consisting of acrylic and/or methacrylic acid homo- or co-polymers, their salts and esters, acrylic acid and acrylamide copolymers, acrylates/C₁₀₋₃₀ alkyl acrylates copolymers, polyacrylamidomethyl propane sulfonic acid and its derivatives, and carbomers; and
cellulose derivatives selected from the group consisting of hydroxypropylcellulose, hydroxyethylcellulose, ethylhydroxyethyl cellulose, methylcellulose, hydroxypropyl methylcellulose, carboxymethyl cellulose, and quaternized derivatives of cellulose;
and mixtures thereof.

13. The composition according to claim 1, wherein the gelling agent is chosen from the group consisting of carbomers and hydroxythylcellulose (HEC), and mixtures thereof.

14. The composition according to claim 1, wherein the total amount of the gelling agent in the composition is from 0.2 to 0.5% by weight, relative to the total weight of the composition.

15. The composition according to claim 1, wherein the foaming surfactant is chosen from the group consisting of:
amphoteric surfactants chosen from the group consisting of
betaine derivatives,
sultaines,
alkyl polyaminocarboxylates,
alkyl amphoacetates, and
imidazole derivatives; and
non-ionic surfactants chosen from the group consisting of
polycondensates of oxyethylene/oxypropylene,
amine derivatives,
silicone derivatives,
saccharide derivatives, and
ethylene oxide derivatives;
and mixtures thereof.

16. The composition according to claim 1, wherein the foaming surfactant is a betaine derivative chosen from the group consisting of alkylbetaines, and alkylaminobetaines, and mixtures thereof.

17. The composition according to claim 1, wherein the total amount of foaming surfactant(s) ranges from 0.5 to 6% by weight, relative to the total weight of the composition.

18. The composition according to claim 1, wherein the composition further contains one or more surfactants chosen from the group consisting of anionic surfactants, cationic surfactants, amphoteric surfactants, and mixtures thereof.

19. The composition according to claim 1, wherein the total amount of surfactant(s) including the foaming surfactant(s) ranges from 0.1 to 10% by weight, relative to the total weight of the composition.

20. The composition according to claim 1, containing further additives and/or active agents chosen from the group consisting of preservatives; preserving agents; sequestering agents; chelating agents; antioxidants; fragrances; pH modifiers; antiseptics; antimicrobials; natural extracts having soothing, moisturizing, and healing properties; soothing agents; and emollients; and mixtures thereof.

21. A method of using the composition according to claim 1 comprising applying the composition to the skin and/or mucous membrane for cleansing.

22. A method of using the composition according to claim 1 comprising applying the composition to tissue paper for cleansing of the skin and/or mucous membrane.

23. A kit for cleansing of the skin and/or mucous membranes comprising the composition according to claim 1 and a dispensing foam pump container.

24. The kit according to claim 23 wherein said kit further comprises tissue paper.

25. A container comprising a dispensing foam pump and the composition according to claim 1.

26. An automatic foam dispenser comprising the composition according to claim 1.

27. A tissue paper product, comprising a composition comprising:
at least 70% by weight of water, relative to the total weight of the composition;
0.1 to 1% by weight of at least one gelling agent, relative to the total weight of the composition, and
0.1 to 10% by weight of at least one surfactant including at least one foaming surfactant, relative to the total weight of the composition.
